(19) United States
(12) Patent Application Publication
(10) Pub. No.: US 2018/0136223 A1
(43) Pub. Date: May 17, 2018

(54) PROCESS FOR EVALUATION OF ABSORPTION CAPACITY OF MYCOTOXINS IN MONOGASTRIC AND POLYGASTRIC ANIMALS

(71) Applicant: BIOVET, S.A., Cambrils (ES)

(72) Inventor: Jaime Valls Borrell, Tarragona (ES)

(73) Assignee: BIOVET, S.A., Cambrils (ES)

(21) Appl. No.: 15/718,472

(22) Filed: Sep. 28, 2017

(30) Foreign Application Priority Data
Nov. 11, 2016 (ES) P201631464

(51) Int. Cl. G01N 33/68

(52) U.S. Cl. CPC G01N 33/6854; G01N 2333/37

(57) ABSTRACT

A process to evaluate absorption of mycotoxins in monogastric and polygastric animals, with the following steps: (a) starting with a sample with mycotoxins on a membrane composed of dextran and polyethylene gels; (b) Adding pepsin to the sample; (c) Adding mycotoxin binder in a part of the sample to be analysed; (d) submitting the sample to several stops in different pH conditions and quantifying the concentration of mycotoxins adsorbed by the binder at each pH condition; (e) repeating each step described in d) with the other part of the sample without mycotoxin binder; and (f) comparing results of adsorption with the part treated with binder against the non treated part of the sample.

### FIELD OF INVENTION

This present invention is about the field of mycotoxins analysis. In particular, the present invention is about a process to evaluate capability of mycotoxins absorption in monogastric and polygastric animals, pretending to simulate conditions in digestive tract with the aim to determine precisely this absorption capacity.

### BACKGROUND OF THE INVENTION

Mycotoxins are fungal metabolites affecting all crops and all livestock, because cereals used in feed manufacturing are contaminated by them.

In industry there are several systems of control and elimination of mycotoxins to avoid them to be absorbed by animals in order to minimize pathogen effects of the same and its incidence in productive parameters. To be able to suggest or decide one system or another to control mycotoxins, some analyses are required about capability of each one of these systems to absorb mycotoxins.

In 1977 the field trials about fixation or adsorption of mycotoxins by Dr. Hernandez (unpublished results) in which an approximation of the digestive structure of monogastric animals is made, studying the contaminated feed by aflatoxins in a system imitating stomach at pH=3.

Latterly in “Newer methods which simulate the G.I.T. (and estimate absorption)” (Doll et al, 2004, Doll and Danicke, 2004 and Avantaggiato et al., 2004) the study is enlarged considering also adsorption at intestinal level, making a wider study at pH=3 and pH=6, corresponding to duodenum. This technique, although more exact does not have into account the last part of intestine, it is, colon.

This last section affects mycotoxin absorption, so that a mycotoxin desorption occurs and they have an entry to the bloodstream through the venous system of superior hemorrhoids that are a ramification of porta vein, through what some mycotoxins pass to porta-hepatic system and other mycotoxins will pass to the system of medium or inferior hemorrhoids, which are a ramification of cava vein, transporting absorbed mycotoxins to the systemic general circulation.

So, there is a need for a more precise procedure that allows the evaluation of the adsorption capacity of mycotoxins in both monogastric animals and polygastric animals in order to avoid them.

The present invention provides a method to meet this need.

### SUMMARY OF THE INVENTION

The present invention is relates to a method for evaluating the ability of adsorption of mycotoxins in monogastric and polygastric animals.

### DETAILED DESCRIPTION OF THE INVENTION

The present invention is about a process to evaluate absorption of mycotoxins in monogastric and polygastric animals, with the following steps:
(a) to have available a sample with mycotoxins on membranes composed of dextran and polyethylene gels;
(b) adding pepsin to the sample;
(c) adding mycotoxin binder in a part of the sample to be analysed;
(d) submitting the sample to several stops in different pH conditions and quantifying the concentration of mycotoxins adsorbed by the binder at each pH condition;
(e) repeating each step described in d) with the other part of the sample without mycotoxin binder; and
(f) comparing results of adsorption with the part treated with binder against the non treated part of the sample.

As an example, a mycotoxin binder can be glucomannans, enzymes, clinoptilolite, silicoglycidol or a combination of these.

This procedure imitates the path of alimentary bolus through the digestive system in animals, referred both to the system of absorption at epithelial level by enterocytes in different parts of intestine that will be simulated with a filtration and different pH values that can be found in a digestive system.

The intestinal skin recovering internal layer of the colon and rectus is formed by several layers allowing permeability to molecules of different molecular weight.

As shown in step a) the proceeding of filtration will imitate these parts of the epithelium is formed by membranes composed of different gels of dextran (Purath, J. y Flodin, P. Nature 183, 1657; 1959) and polyethylene (Moore, J; J. Polym. Sci, 2,835; 1964) that are able to absorb amino acids, fatty acids and hydrolysed carbohydrates during digestion.

In the first section of intestinal epithelium amino acids are absorbed with a molecular weight between 83 and 291 (lysine 146.19, methionine 143.21, leucine 149.21), such as fatty acids (butyric acid 88.11, stearic acid 281.48 and oleic acids 288.3) and simple sugars (glucose 180.1, fructose 180.16). All of these come from digestion of nutritional principles of food. The above numbers refer to the molecular weight of the mentioned compound.

In the second section of intestinal epithelium molecules with molecular weight between 291 and 833 are absorbed. Mainly this section will absorb non fixed mycotoxins to binder (Deoxinivalenol 296, zearalenone 318, aflatoxines 312-330 except G2 with a molecular weight of 346, ocratoxin 403 and fumonisin 721) and enzymes (sacarase 342.29). The above numbers refer to the molecular weight of the mentioned compound.

In the third section of intestinal epithelium the complex molecules with molecular weight between 833 and 12500 are absorbed. These are the resistant molecules to gastric juices such as subunits of digestive enzymes (peptidases) and simple components of starch (amylose).

In a exemplary embodiment of the invention, these animals are monogastric. As “monogastric” it is understood to be animals with a simple stomach, with storage capacity similar to a human. Monogastric animals do not make a pregastric fermentation, although some, such as rabbits or horses, make a postgastric fermentation, thanks to the functional caecum, that have microorganisms inside and are able to digest fiber portions (cellulose and hem icellulose). Other examples of monogastric animals include omnivores such as humans, rats, swine, carnivores such as dogs and cats and herbivores such as horses and rabbits.

The processes of the present invention include differences between monogastric animals and polygastric ones, because digestive conditions to be reproduced are different.

In another exemplary embodiment of the present invention, in the case of monogastric animals, conditions to be reproduced in step d) will be at pH 3, at pH 5 and at pH 9.

In another exemplary embodiment of the present invention, in the case of polygastric animals, also called ruminants, they have a complex anatomic structure formed by four parts: rumen, omasum, abomasum and reticulum. The first three are called pre-stomach and have an aglandular mucosa (which is an epithelium without the skill to produce juices with digestive function).

In another exemplary embodiment of the present invention, in the case of polygastric animals, the conditions of pH to be reproduced in step d) will be at pH 6.1, at pH 2.5, at pH 7.5, at pH 4.2 and at pH 7.

In another exemplary embodiment of the present invention, quantification of the mycotoxins concentration in the comparison step number f) should be done by ELISA. In other exemplary embodiments, other quantifications may be utilized.

In another exemplary embodiment of the present invention, step d) is made as follows for monogastric animals:
(d1) Adsorption at pH 3 is made with an aqueous solution formed by disodium phosphate (Na₂H₂PO₄) 1M and phosphoric acid (H₃PO₄) 0.1 M (it simulates stomach pH) and mycotoxin binder at a concentration of 0.05% w/w. It must be shaken for 2 hours between 38 and 42° C. After a centrifugation (10 min, 4,500 r.p.m.) an aliquot of 15 ml of sample is taken;
(d2) Then, the pH of the sample is changed to pH 5 using an adequate base, such as sodium bicarbonate. Sample is shaken for 2 hours at 38° C. and centrifugation (10 min, 4,500 r.p.m.) to 15 ml of the sample; and
(d3) Finally to get pH 9 an adequate base is used such as, for example, sodium bicarbonate. Sample is shaken for 2 hours at 38° C. and centrifugation (10 min, 4,500 r.p.m.) to 15 ml of the sample.

In another exemplary embodiment of the present invention, in the case of polygastric animals, the procedure for step d) is as follows:
(d1) Adsorption at pH 6.1 is made with an aqueous solution, a buffer at 6.1 and a mycotoxin binder at a concentration 0.05% w/w. It is mixed for 2 hours at 42° C. After a centrifugation is carried out (10 min, 4,500 r.p.m.) to 15 ml of the aliquot of the sample;
(d2) then, the pH is adjusted to pH 2.5 using an adequate acid, for example HCl. Sample will be shaken for 2 hours at 42° C. After, a centrifugation is carried out (10 min, 4,500 r.p.m.) to 15 ml of the aliquot of the sample;
(d3) then, the pH is adjusted to 7.5 using an adequate base, such as sodium bicarbonate. It is mixed for 2 hours at 38° C. After, a centrifugation is carried out (10 min, 4,500 r.p.m.) to 15 ml of the aliquot of the sample;
(d4) then, the pH is adjusted to pH 24.2 using an adequate acid, such as, for example HCl. The sample is shaken for 2 hours at 42° C. After, a centrifugation is carried out (10 min, 4,500 r.p.m.) to 15 ml of the aliquot of the sample;
(d5) Finally, the pH is adjusted to 7 using an adequate base, such as sodium bicarbonate. It is mixed for 2 hours at 38° C. After, a centrifugation is carried out (10 min, 4,500 r.p.m.) to 15 ml of the aliquot of the sample.

Following, some examples are shown, but with no aim to limit the invention.

### Examples

1. Simulation of the Study of Absorption of Mycotoxin Binders in Monogastric Animals.

A solution of distilled water is prepared with a quantity of mycotoxins considered appropriate (for example 300 ppb of aflatoxins, 1 ppm of zearalenone or fumonisin B1 in alcoholic solution). Pepsin is also added to a solution containing mycotoxins at a concentration of 17.331 international units (IU) per litre.

The sample is divided into two parts. One will be analysed without any other treatment, so it will act as blank/control/not treated sample. To the second half of the sample, 0.5 g/litre of a mycotoxin binder is tested/evaluated and this is referred to hereinafter as the treated sample.

As in an exemplary digestive tract of monogastric animals, pH changes from 3 to 5 and finally to 7, we can reproduce these conditions by adjusting pH.
(1) In a first place, and for both parts of the sample (the blank and the one with mycotoxin binder) HCl (hydrochloric acid) is used to achieve pH 3. Take 15 ml of the blank and another of the treated solution and shake them for 2 hours at a temperature between 38 and 42° C. Centrifugate each sample for 10 minutes at 4,500 rpm. Take the supernatant that contains free mycotoxins (those that were not absorbed by the binder) and a quantification using ELISA is performed.

The result of is calculated as concentration of free mycotoxins of the blank-concentration of free mycotoxins in the sample with binder.
(2) The samples are taken and using NaHCO₃ 0.5 M (monosodium carbonate) are taken to pH 5. Take 15 ml of the blank and another of the treated sample and centrifuge them for 10 minutes at 4,500 rpm. Take the supernatant that contains free mycotoxins (those that were not absorbed by binder) and quantification using ELISA is performed.

The result is calculated as in step 1
(3) The samples are taken and using NaHCO₃ 0.5 M (monosodium carbonate) are taken to pH 9. Take 15 ml of the blank and another of the treated sample and centrifuge them for 10 minutes at 4,500 rpm. Take the supernatant that contains free mycotoxins (those that were not absorbed by binder) and quantification using ELISA is performed.

The result is calculated as in step 1

The obtained results of binding mycotoxins for a concentration of fumonisin 2 ppm and a dose of mycotoxin binder of 0.5 g/kg are:

| | Binding | % | Desortion | % |
|---|---|---|---|---|
| | (μg/l) | adsorption | (μg/l) | desortion |
| pH 3 (stomach) | 1999.2 | 99.96 | 0.8 | 0.04 |
| pH 5 (duodenum) | 1801.28 | 90.10 | 197.92 | 9.86 |
| pH 9 (rectum) | 1668.89 | 83.43 | 132.39 | 6.62 |

% adsorption of all digestive tract: 83.48%

2. Simulation of the Study of Absorption of Mycotoxin Binders in Polygastric Animals

A solution of distilled water is prepared with a quantity of mycotoxins considered appropriate (for example 300 ppb of aflatoxins, 1 ppm of zearalenone or fumonisin B1 in alcoholic solution). Pepsin is also added to a solution containing mycotoxins at a concentration of 17.331 international units (IU) per litre.

The sample is divided into two parts. One will be analysed without any other treatment, so it will act as blank. To the second half of the sample, 0.5 g/litre of a mycotoxin binder to be tested.

As in an exemplary digestive tract of polygastric animals, pH changes from 6.1 to 2.5, changes again to 7.5 to decrease again to 2.5, and finally to 7.5, these conditions can be reproduced by adjusting pH.
(1) Firstly, NaHCO₃ 0.5 M to buffer the solution at pH 6.1. Stir for 2 hours at 38-42° C. Take 15 ml of the blank and another of the treated sample and centrifuge them for 10 minutes at 4,500 rpm. Take the supernatant that contains free mycotoxins (those that were not absorbed by binder) and quantification using ELISA is performed.

The result is calculated as in step 1.
(2) The pH of the samples is now taken to 2.5 using HCl (hydrochloric acid). Take 15 ml of the blank and another of the treated sample and centrifuge them for 10 minutes at 4,500 rpm. Take the supernatant that contains free mycotoxins (those that were not absorbed by binder) and quantification using ELISA is performed.

The result is calculated as in step 1.
(3) In this step pH is increased up to 7.5 by using a buffer solution of NaHCO₃ 0.5 M. Take 15 ml of the blank and another of the treated sample and centrifuge them during 10 minutes at 4,500 rpm. Take the supernatant that contains free mycotoxins (those that were not absorbed by binder) and quantification using ELISA is performed.

The result is calculated as in step 1.
(4) The pH sample is now modified to 4.2 using HCl (hydrochloric acid). Take 15 ml of the blank and another of the treated sample and centrifuge them during 10 minutes at 4,500 rpm. Take the supernatant that contains free mycotoxins (those that were not absorbed by binder) and quantification using ELISA is performed.

The result is calculated as in step 1.
(5) Finally, pH is modified up to pH 7 using a buffer solution of NaHCO₃ 0.5 M. Take 15 ml of the blank and another of the treated sample and centrifuge them for 10 minutes at 4,500 rpm. Take the supernatant that contains free mycotoxins (those that were not absorbed by binder) and quantification using ELISA is performed.

The result is calculated as in step 1.

The obtained results of binding for different binders for a concentration of aflatoxin B1 300 ppb and a dose of mycotoxin binder of 0.5 g/kg are:

| | pH 6.1 | pH 2.5 | pH 7.5 | pH 4.2 | pH 7 |
|---|---|---|---|---|---|
| Glucomannans | 21.66% | 20.23% | 13.73% | 17.73% | 42.12% |
| Enzymes | 99.91% | 100% | 99.06% | 100% | 100% |
| Clinoptilolita | 25.81% | 67% | 61% | 63.70% | 60.4% |
| Silicoglicidol | 95.67% | 100% | 100% | 63.70% | 100% |

According to the table, it may be observed that with the process of this invention, enzymes and silicoglycidol have the best mycotoxins binding skill, followed by clinoptilolite and finally glucomannans.

1. A process for evaluating adsorption capacity in monogastric and polygastric animals with the following steps:
(a) having a sample with mycotoxins on membrane composed of dextrane and polyethylene gels;
(b) adding pepsin to the sample;
(c) adding mycotoxin binder in a part of the sample to be analysed;
(d) submitting the sample to several stops in different pH conditions and quantifying concentration of mycotoxins adsorbed by the binder at each pH condition;
(e) repeating each step described in d) with the other part of the sample without mycotoxin binder; and
(f) comparing results of adsorption with the part treated with binder against the non treated part of the sample.

2. The process of claim 1, wherein said sample is from a monogastric animal.

3. The process of claim 1, wherein said binding absorption is at pH 3, at 5 and at 9.

4. The process of claim 1, wherein said sample is from a polygastric animal.

5. The process of claim 1, wherein pH conditions for step d) are at 6.1, then at 2.5, next at 7.5, then at 4.2 and finally at 7.

6. The process of claim 1, using ELISA method of mycotoxins quantification.

7. The process of claim 1, wherein in step f) by rest of said mycotoxin concentration of the non treated part of the sample and concentration of free mycotoxins in the sample with binder.

8. The process of claim 1, wherein the mycotoxins binder added in step (c) is either a glucomannans, enzymes, clinoptilolite, silicoglycidol or a mix of some of them.
